(19) United States
(12) Patent Application Publication
(10) Pub. No.: US 2005/0095212 A1
(43) Pub. Date: May 5, 2005

(54) Hair cosmetic compositions

(75) Inventor: Yuji Hirano, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kao Corporation, Tokyo (JP)

(21) Appl. No.: 10/694,774

(22) Filed: Oct. 29, 2003

(51) Int. Cl. A61K 7/06; A61K 7/11

(52) U.S. Cl. 424/70.11

(57) ABSTRACT

Hair cosmetic compositions contain (A) a film-forming polymer; and (B) a diamide compound represented by the following formula (1): 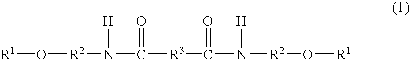
wherein R¹ represents a linear or branched C₁₋₁₂ hydrocarbon group which may be substituted by one or more hydroxyl groups and/or alkoxy groups, R² represents a linear or branched, divalent C₁₋₅ hydrocarbon group, and R³ represents a linear or branched, divalent C₁₋₂₂ hydrocarbon group. The hair cosmetic compositions have for example excellent hair styling properties, may protect hair from physical and chemical stimulation to inhibit occurrence of split ends or broken hair and further, may impart a good feel to hair.

### FIELD OF THE INVENTION

This invention relates to hair cosmetic compositions containing amide compounds, which are for example excellent in hair styling properties, may protect hair from physical and chemical damages to inhibit occurrence of split ends or broken hair and further, may impart a good feel to hair.

### BACKGROUND OF THE INVENTION

Hair exists in a damaged state accompanied by a partial loss of its components and structural elements, because it is often exposed to physical damage due to everyday hair care activities, such as dryer heat and brushing friction, and also to chemical damages by permanent wave preparations, hair colors, hair bleaches and/or the like. From the viewpoint of hair care, it is not preferred to leave hair in such a damaged state as it leads to the occurrence of split ends and broken hair, a dry and loose feel, an untidy hairstyle and the like. Protection and/or repair of damaged hair is generally conducted in a way such as supplementing a component or structural element, which has been lost due to the damage, with the corresponding component or structural element or with an analogous substance thereof. For the development of a protecting and/or repairing function, an interaction (compatibility) between a protecting base and hair is considered to be important, and at present, a method making use of a sphingo-lipid or a protein derivative as a protecting base has found wide-spread utility as a beneficial technique. It is, however, the current circumstance that such a base may not be added in any sufficient amount to products from a cost-related consideration despite its recognized effects since the production of such a base requires the need to go through extremely complex steps.

### SUMMARY OF THE INVENTION

In one aspect of the present invention, there is thus provided a hair cosmetic composition which contains the following ingredients (A) and (B):
(A) a film-forming polymer; and
(B) a diamide compound represented by the following formula (1): 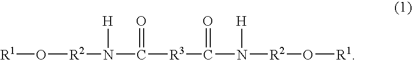
   R¹ represents a linear or branched C₁₋₁₂ hydrocarbon group which may be substituted by one or more hydroxyl groups and/or alkoxy groups, R² represents a linear or branched, divalent C₁₋₅ hydrocarbon group, and R³ represents a linear or branched, divalent C₁₋₂₂ hydrocarbon group.

### DETAILED DESCRIPTION OF THE INVENTION

The present invention relates to a hair cosmetic composition containing a base, which may be produced readily and may be supplied at low cost and is effective for the protection and/or repair of hair, having excellent hair styling properties, and capable of giving an excellent feel in use.

The present inventors have found that certain diamide compounds of a specific structure, which may be produced readily at low cost, have excellent effects for inhibiting split ends and broken hair and further, such hair cosmetic compositions making use of these diamide compounds in combination with film-forming polymers are excellent in both hair styling properties and feel in use.

The film-forming polymer employed as the ingredient (A) in the present invention may be anionic, amphoteric, cationic or nonionic.

Illustrative anionic film-forming polymers include lower alkyl ester of methyl vinyl ether/maleic acid copolymers (“Gantrez ES-225”, “Gantrez ES-425” and “Gantrez SP-215”, trade names, products of International Specialty Products Corporation; etc.), acrylic acid/ethyl acrylate/N-(t-butyl)acrylamide copolymers (“Ultrahold 8” and “Ultrahold Strong”, trade names, products of BASF AG; etc.), octylacrylamide/acrylic acid copolymers (“Amphomer V-42”, trade name, product of National Starch & Chemical Company; etc.), vinyl acetate/crotonic acid copolymers (“Resyn 28-1310”, trade name, product of National Starch & Chemical Company; etc.), vinyl acetate/crotonic acid/vinyl neodecanoate copolymers (“Resyn 28-2930”, trade name, product of National Starch & Chemical Company; etc.), vinyl acetate/crotonic acid/vinyl propionate copolymers (“Luviset CAP”, trade name, product of BASFAG; etc.), water-dispersible polyesters (“AQ38S” and “AQ55S”, trade names, products of Eastman Kodak Company; etc.), acrylate/methacrylate/acrylic acid/methacrylic acid copolymers (“Amerhold DR-25”, trade name, Union Carbide Corporation; etc.), alkyl acrylate/diacetone acrylamide copolymers AMP (“Plas size L9540B”, trade name, product of GOO CHEMICAL CO., LTD.; etc.), vinyl alcohol/itaconic acid copolymers (“KM-118”, trade name, product of Kuraray Co. , Ltd., etc.), polyacrylic acids (“Carbopol 910”, “Carbopol 940” and “Carbopol 980”, trade names, products of BF Goodrich Co.; etc.), carageenan (“Soageena LX22” and “Soageena ML210”, trade names, products of Mitsubishi Rayon Co., Ltd.; etc.), and xanthan gum (“Echogum T”, trade name, product of Dainippon Pharmaceutical Co., Ltd.; etc.).

Illustrative amphoteric film-forming polymers include dimethyldiallylammonium chloride/acrylamide/acrylic acid copolymers (“MERQUAT 3331”, trade name, product of Calgon Corp., etc.), (meth)acrylethylbetaine/alkyl(meth)acrylate copolymers such as N-methacryloyloxyethyl-N,N-dimethylammonium-α-N-methylcarb oxybetaine/alkyl methacrylate copolymers (“Yukaformer M-75” and “Yukaformer SM”, trade names, products of Mitsubishi Chemical Corporation; etc.); and alkyl acrylate/butylaminoethyl methacrylate/octyl acrylate amide copolymers such as octylacrylamide/acrylate/butylaminoethyl methacrylate copolymers (“Amphomer 28-4910”, trade name, product of National Starch & Chemical Company; etc.).

Illustrative cationic film-forming polymers include polydimethyldiallylammonium chloride (“MERQUAT 100”, trade name, product of Calgon Corp.; etc.), acrylamidopropyltrimethylammonium chloride/acrylate copolymers (“MERQUAT”, trade name, product of Calgon Corp.; etc.), acrylamide/dimethyldiallylammonium chloride copolymers (“MERQUAT 550” and “MERQUAT 2220”, trade names, products of Calgon Corp.; etc.), t-butylacrylamide/ethyl acrylate/dimethylaminopropylacrylamide/methoxypolyethylene glycol methacrylate copolymers (“RP77S”, trade name, product of Kao Corporation; etc.), t-butylacrylamide/dimethylacrylamide/dimethylaminopropylacrylamide/methoxypolyethylene glycol methacrylate copolymers (“RP77T”, trade name, product of Kao Corporation; etc.), methylvinylimidazolium chloride/vinylpyrrolidone copolymers (“Luviquat FC370”, “Luviquat FC550”, “Luviquat FC905” and “Luviquat HM552”, trade names, products of BASF AG; etc.), vinylpyrrolidone/dimethylaminopropylmethacrylamide copolymers (“Gafquat HS-100”, trade name, product of International Specialty Products Corporation.; etc.), diethyl sulfates of vinylpyrrolidone/dimethylaminoethyl methacrylate copolymers (“Gafquat 734”, “Gafquat 755N” and “Gafquat 755”, trade names, products of International Specialty Products Corporation; etc.), vinyl alcohol/dimethylaminopropylmethacrylamide copolymers (“C-318”, trade name, product of Kuraray Co., Ltd.; etc.), vinylpyrrolidone/dimethylaminoethyl methacrylate copolymers (“Copolymer 845”, “Copolymer 937” and “Copolymer 958”, trade names, products of International Specialty Products Corporation; etc.), vinylpyrrolidone/alkylaminoacrylate/vinylcaprolactam copolymers (“Copolymer VC-713”, trade name, product of International Specialty Products Corporation; etc.), hydroxyethylcellulose/dimethyldiallylammonium chloride copolymers (“Celquat H-100” and “Celquat L-200”, trade names, products of National Starch & Chemical Company; etc.), hydroxyethylcellulose/2-hydropropyltrimethylammonium chloride copolymers (“Polymer JR-400”, trade name, product of Union Carbide Corporation; etc.), guar hydroxypropyltrimonium chloride (“Cosmedia Guar C-261N”, trade name, product of Henkel Corporation, “Jaguar C-17”, trade name, product of Rhône-Poulanc S.A., etc.), and aminoalkyldimethylpolysiloxane/polyethylenoxazoline copolymers disclosed in EP-A-640,643.

Illustrative nonionic film-forming polymers include polyvinylpyrrolidone (“Luviskol K-12”, “Luviskol K-30” and “Luviskol PVP K-120”, trade names, products of BASF AG), polyvinyl alcohol (“Gohsenol EG-05” and “Gohsenol EG-20”, trade names, products of The Nippon Synthetic Chemical Industry Co., Ltd.; etc.), vinyl alcohol/vinylamine copolymers (“VA-120-HCl”, trade name, product of Air Products & Chemicals, Inc.; etc.), vinylpyrrolidone/vinyl acetate copolymers (“Luviskol VA28” and “Luviskol VA64W”, trade names, products of BASF AG, “VA73”, “PVP/VA E-535” and “PVP/VA E-735”, trade names, products of International Specialty Products Corporation; etc.), acrylate/vinylpyrrolidone copolymers (“Luviflex VBM35”, trade name, product of BASF AG), polyethylene glycol of high polymerization degree (“POLYOX WSRN-60K”, trade name, product of Union Carbide Japan K.K.; etc.), guargum (“Fibalon S”, trade name, product of Dainippon Pharmaceutical Co., Ltd.; etc.), pullulan (“PF-10”, trade name, product of Hayashibara Biochemical Laboratories, Inc.; etc.), hydroxypropyl chitosan (“Chitofilmer HV-10”, trade name, product of ICHIMARU PHARCOS Co., Ltd.; etc.), and chitosan-dl-pyrrolidonecarboxylate salts (“Chitomer PC”, trade name, product of Union Carbide Corporation).

Among the film-forming polymers described above, particularly preferred are the anionic or amphoteric film-forming polymers containing (meth)acrylic acid units, (meth)acrylate units or vinyl acetate units; the cationic film-forming polymers containing dimethyldiallyl ammonium units or dimethylaminoalkyl(meth)acrylamide units; the copolymers disclosed in EP-A-640, 643; the nonionic film-forming polymers containing vinylpyrrolidone units; the cationized cellulose; carageenan; and the chitosan derivatives.

Two or more film-forming polymers may be used in combination as the ingredient (A). The content of the ingredient (A) may range preferably from 0.05 to 30 wt. %, more preferably from 0.1 to 20 wt. %, even more preferably from 0.2 to 10 wt. %, all in terms of solids based on the whole component (in the case of an aerosol or foam, it is based on the concentrate; this will equally apply hereinafter in the description).

In formula (1) representing the diamide compound employed as the ingredient (B) in the present invention, R¹ may preferably be a linear or branched C-₁₋₁₂ alkyl group which may be substituted by 1 to 3 substituents selected from hydroxyl groups and C₁₋₆ alkoxy groups. Among such alkyl groups, more preferred are unsubstituted C₁₋₁₂ alkyl groups and C₂₋₁₂ alkyl groups each of which is substituted by 1 to 2 hydroxyl groups, one C₁₋₆ alkoxy group, or one hydroxyl group and one C₁₋₆ alkoxy group. Specific examples include methyl, ethyl, propyl, butyl, hexyl, dodecyl, 2-methylpropyl, 2-ethylhexyl, 2-hydroxyethyl, 9-hydroxynonyl, 2,3-dihydroxypropyl, 2-methoxyethyl, 2-hydroxy-3-methoxypropyl, and 9-methoxynonyl. Among these, 2-hydroxyethyl, methyl, dodecyl and 2-methoxyethyl are preferred.

In formula (1), R² may preferably be a linear or branched C₂₋₅, more preferably a C₂₋₃ alkylene group. Specific examples include ethylene, trimethylene, tetramethylene, pentamethylene, 1-methylethylene, 2-methylethylene, 1-methyltrimethylene, 2-methyltrimethylene, 1,1-dimethylethylene, and 2-ethyltrimethylene. Among these, ethylene and trimethylene are preferred.

In formula (1), R³ may preferably be a linear or branched, divalent C₂₋₂₂ hydrocarbon group, with a linear or branched C₁₁₋₂₂ alkylene group or a linear or branched C₁₁₋₂₂ alkenylene group having 1 to 4 double bonds being particularly preferred. Specific examples include ethylene, trimethylene, tetramethylene, hexamethylene, heptamethylene, octamethylene, decamethylene, undecamethylene, dodecamethylene, tridecamethylene, tetradecamethylene, hexadecamethylene, octadecamethylene, 1-methylethylene, 2-ethyltrimethylene, 1-methylheptamethylene, 2-methylheptamethylene, 1-butylhexamethylene, 2-methyl-5-ethylheptamethylene, 2,3,6-trimethylheptamethylene, 6-ethyldecamethylene, 7-methyltetradecamethylene, 7-ethylhexadecamethylene, 7,12-dimethyloctadecamethylene, 8,11-dimethyloctadecamethylene, 7,10-dimethyl-7-ethylhexadecamethylene, 1-octadecylethylene, ethenylene, 1-octadecenylethylene, 7,11-octadecadienylene, 7-ethenyl-9-hexadecamethylene, 7,12-dimethyl-7,11-octadecadienylene, and 8,11-dimethyl-7,11-octadecadienylene. Among these, 7,12-dimethyloctadecamethylene, 7,12-dimethyl-7,11-octadecadienylene, octadecamethylene, undecamethylene and tridecamethylene are particularly preferred.

Diamide compounds particularly preferred as the ingredient (B) are those containing the above-exemplified, preferred groups as R¹, R² and R³ in formula (1) in combination. Particularly preferred specific examples of the diamide compound (1) include the following compounds:

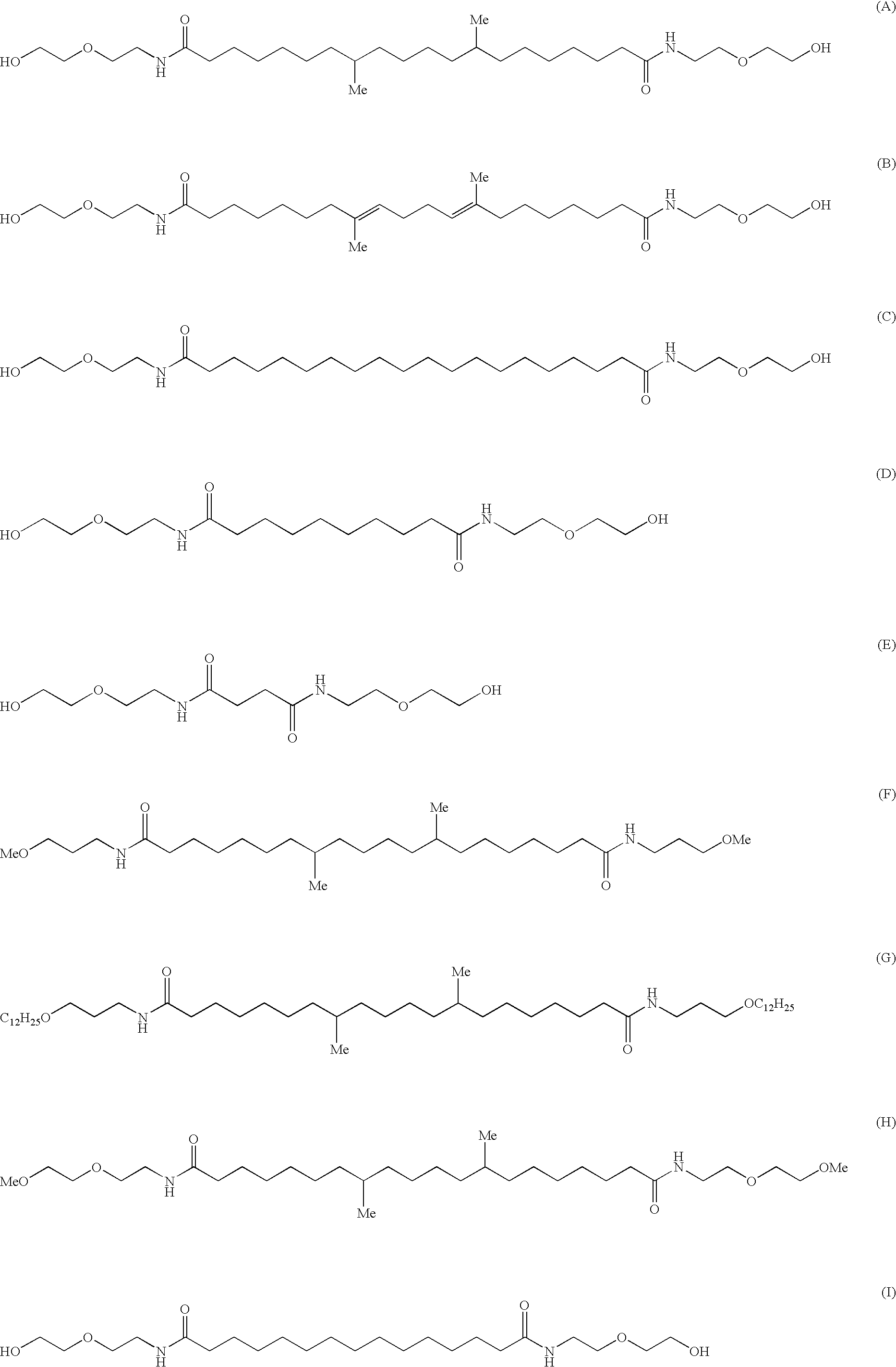

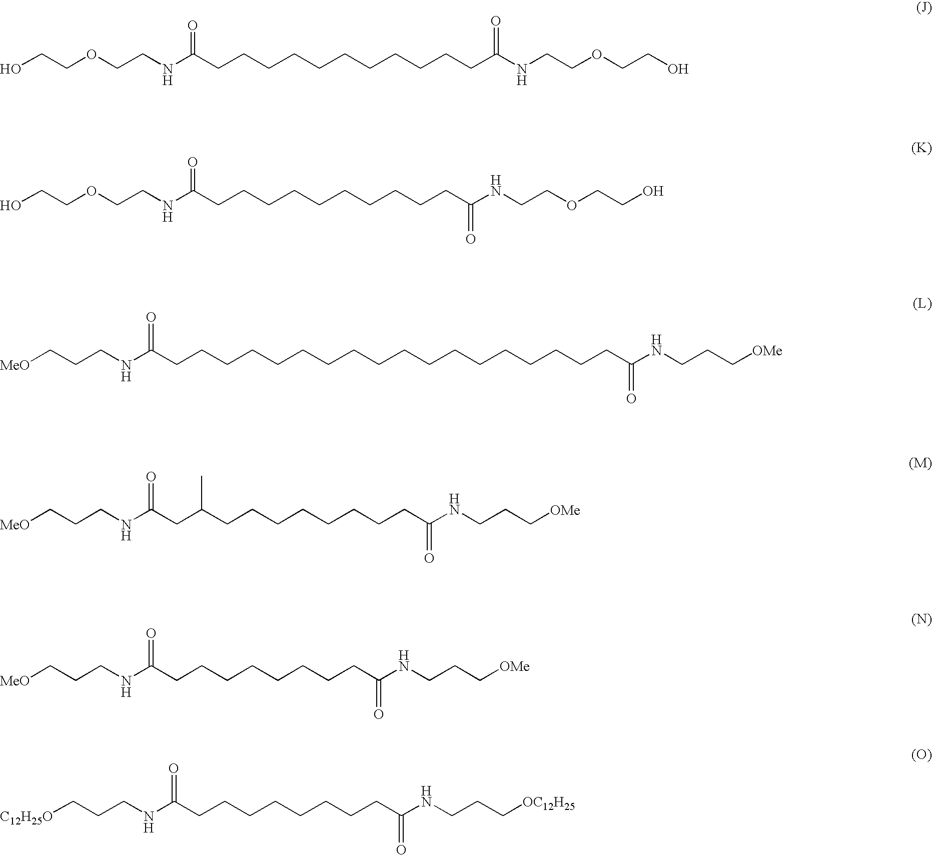

As the ingredient (B), two or more diamide compounds (1) may be used in combination. The content of the ingredient (B) may range preferably from 0.01 to 20 wt. %, more preferably from 0.1 to 20 wt. %, even more preferably from 0.5 to 15 wt. %, all based on the whole composition. Incidentally, these diamide compounds (1) can be synthesized by the process disclosed in the international publication WO 00/61097.

For the purpose of further improving the effects for inhibiting split ends and broken hair, one or more of proteins and ceramides, each of which is commonly employed as a hair protecting ingredient, may be included in the hair cosmetic composition according to the present invention.

Such proteins type include proteins, protein hydrolysates and derivatives thereof, and can be obtained by extraction from animals or plants or by deriving from the proteins so extracted. Examples of proteins of animal origin include keratin, elastine, collagen, lactoferin, casein, α(β)-lactoalbumin, globulins, ovalbumin, silk protein and hydrolysates thereof, with keratin, elastine, collagen, casein, silk protein and hydrolysates thereof being preferred. Examples of proteins of plant origin, on the other hand, include proteins extracted from wheat, malt, oat, barley, corn, rice, soybeans, broad beans, lupine seeds, potatoes and apricot kernels, and hydrolysates thereof, with wheat protein, soybean protein and hydrolysates thereof being preferred. Two or more proteins may be used in combination. The content of protein(s) may range preferably from 0.01 to 5 wt. %, more preferably from 0.05 to 4 wt. %, even more preferably from 0.1 to 3 wt. %, all based on the whole composition.

Such ceramides include N-acylated sphingosines, N-acylated phytosphingosines and N-acylated dihydrosphingosines, all of which are available by synthesis or extraction from natural sources. Substituent groups in the acyl substituent on sphingosine, dihydrosphingosine and phytosphingosine can be linear or branched, C₈₋₂₂ alkyl or alkenyl groups, 1 to 5 hydrogen atoms of each of which may be substituted by a like number of hydroxyl groups. For example, in addition to Ceramide 1, Ceramide 2, Ceramide 3, Ceramide 1A, Ceramide 6II and hydroxycaproylphytosphingosine, synthetic pseudo-ceramides such as Sphingo-lipid EX (JP-A-11-209248) and Sphingo-lipid E (JP-B-01-042934) are also usable. Two or more ceramides may be used in combination. The content of ceramide(s) may range preferably from 0.01 to 5 wt. %, more preferably from 0.05 to 4 wt. %, even more preferably from 0.1 to 3 wt. %, all based on the whole composition.

To further improve the feel in use, the hair cosmetic composition according to the present invention may additionally contain one or more silicone derivatives which are commonly employed as ingredients to improve the feel.

Such silicone derivatives include dimethylpolysiloxane, methylphenylpolysiloxane, amino-modified silicones, polyether-modified silicones, epoxy-modified silicones, fluorine-modified silicones, cyclic silicones, alkyl-modified silicones, oxazoline-modified silicones, and the like. Among these, preferred are dimethylpolysiloxane, methylphenylpolysiloxane, amino-modified silicones, polyether-modified silicones, oxazoline-modified silicones, and cyclic silicones. Two or more silicone derivatives may be used in combination. The content of silicone derivative(s) may range preferably from 0.01 to 20 wt. %, more preferably from 0.05 to 10 wt. %, even more preferably from 0.1 to 5 wt. %, all based on the whole composition.

By conventional procedure, the hair cosmetic composition according to the present invention may be formulated into-various preparation forms, specifically into sprays, mists, foams, gels, lotions, tonics, blow styling aids, creams, waxes, post-foaming gels, and so on. When formulating into aerosols or foams, it is possible to include one or more of various propellants such as volatile hydrocarbons such as butane, isobutane, pentane and isopentane; halogenated hydrocarbons such as dichlorofluoromethane, dichlorotetrafluoroethane and 1,1-difluoroethane; compressed gases such as carbon dioxide, nitrogen and air; and dimethyl ether. When formulating into the form of foams, it is possible to include one or more of surfactants such as linear or branched alkylbenzenesulfonate salts, alkyl or alkenyl ether sulfates added with ethylene oxide and/or propylene oxide, olefinsulfonate salts, alkanesulfonate salts, saturated or unsaturated fatty acid salts, alkyl or alkenyl ether carboxylate salts added with ethylene oxide and/or propylene oxide, α-sulfofatty acid esters, amino acid-type surfactants, phosphate-type surfactants, sulfosuccinic acid-type surfactants, sulfonic acid-type surfactants, amphoteric surfactants, betaine-type surfactants, alkylamine oxides, cationic surfactants such as linear or branched, alkyl or alkenyl quaternary ammonium salts, polyoxyalkylene alkyl or alkenyl ethers, polyoxyalkylene alkyl phenyl ethers, higher fatty acid alkanolamides or alkylene oxide adducts thereof, polyhydric alcohol-fatty acid esters, sorbitol-fatty acid esters, sucrose-fatty acid esters, and higher alcohol-sucrose ethers.

In addition to the above-described ingredients, the hair cosmetic composition according to the present invention may further contain, depending on its application purpose, one or more of organic solvents such as C₁₋₆ alcohols as a solvent for the film-forming polymer; oil ingredients such as higher alcohols, fatty acids and salts thereof, cholesterol and derivatives thereof, vaseline, lanolin derivatives, and polyethylene glycol fatty acid esters; polyhydric alcohols such as glycerin and sorbitol; humectants; chelating agents such as ethylenediaminetetraacetic acid (EDTA); medicaments such as vitamins; amino acids and derivatives thereof; fine powders of polymers such as polyethylene, polystyrene, poly(methyl methacrylate), nylon and silicones, and hydrophobicization-treated products thereof; animal and plant extracts; ultraviolet absorbers; pearlants; preservatives; antimicrobial agents; anti-inflammatories; antidandruff agents; pH adjusters; dyestuffs; perfumes; and the like.

### EXAMPLES 1-3 & COMPARATIVE EXAMPLE 1

The materials shown in Table 1 were mixed to provide concentrates for aerosol hair sprays. After those concentrates were filled in suitable pressure containers, respectively, the pressure containers were filled with a propellant (LPG, 2.5 kg/cm² ) at a concentrate:propellant ratio (by weight) of 45:55 to obtain hair sprays.

Those hair sprays were evaluated with regard to unstiffness and the percent occurrence of split ends and broken hair. The results are shown in Table 1.

<Evaluation Methods>

-Stiffness

For each of the hair sprays, predetermined hairstyling was performed on five expert panelists, and the hair spray was applied in an appropriate amount. With respect to the feel of the hair so finished, organoleptic evaluation was performed in accordance with the following standards, and the results were indicated by an average score.

5: Not stiff

4: Not very stiff

3: Hard to say either way

2: A little stiff

1: Stiff

-Percent Occurrence of Split Ends and Broken Hair

1) Flattened hair tresses, each of which was 16 cm in length and was composed of 100 strands of hair, were prepared.

2) With respect to each of the hair sprays, one of the hair tresses was evenly sprayed with the hair spray in an appropriate amount [hair:spray (including the propellant)=1:1 (by weight)]. The tress was then washed with a commercial shampoo, followed by drying in air. Taking the foregoing procedure as a one-time treatment, it was repeated 14 times (equivalent to 12 weeks in actual life).

3) Brushing stimulation was applied to the thus-treated tress by a motor-driven rotary brush at 100 rpm for about 60 minutes.

4) The tress was visually observed for tip conditions, and split ends and broken hair which occurred were counted.

5) Assuming that the percent occurrence of split ends and broken hair in a standard tress (Comparative Example 2) was 100%, the percent inhibitions of split ends and broken hair by the hair spray was determined in accordance with the following formula:

Percent occurrence of split ends and broken hair=(Number of split ends and broken hair occurred in the treated hair tress/number of split ends and broken hair occurred in the standard tress)×100

TABLE 1

| | | | | Comp. |
|---|---|---|---|---|
| (Wt. %) | Ex. 1 | Ex. 2 | Ex. 3 | Ex. 1 |
| Acrylamide/alkyl acrylate/ | 6.0 | 6.0 | 6.0 | 6.0 |
| methoxypolyethylene glycol | | | | |
| methacrylate copolymer¹⁾ | | | | |
| Phosphoric acid (75 wt. %) | 0.5 | 0.5 | 0.5 | 0.5 |
| Diamide compound (F) | 2.0 | 3.0 | 2.0 | — |
| Cetyloxypropyl glyceryl | — | — | 0.5 | — |
| methoxypropyl myristamide²⁾ | | | | |
| Ethyl alcohol | Balance | Balance | Balance | Balance |
| Stiffness | 4.0 | 4.6 | 4.2 | 2.8 |
| Percent occurrence of split ends | 51 | 31 | 39 | 100 |
| and broken hairs | | | | |

¹⁾“RP77S”, trade name; product of Kao Corporation
²⁾“AQUACERAMIDE”, trade name; product of Kao Corporation

### EXAMPLES 4-6 & COMPARATIVE EXAMPLE 2

The materials shown in Table 2 were mixed to provide concentrates for hair foams. After those concentrates were filled in suitable containers, respectively, the containers were filled with a propellant (LPG, 5.0 kg/cm²) at a concentrate:propellant ratio (by weight) of 90:10 to obtain hair foams.

Those hair foams were evaluated with regard to silkiness, moisturized feel and the percent occurrence of split ends and broken hair. The results are shown in Table 2.

<Evaluation Methods>

-Feel to the Touch (Silkiness and Moisturized Feel)

With respect to each of the hair foams in Table 2, predetermined hairstyling was performed on five expert panelists by using the hair foam in an appropriate amount. With respect to the silkiness and moisturized feel of the thus-styled hair, organoleptic evaluation was performed in accordance with the following standards, and the results were indicated by average scores.

(Silkiness)

5: Silky

4: A little silky

3: Hard to say either way

2: Not very silky

1: Not silky

(Moisturized Feel)

5: Moisturized

4: A little moisturized

3: Hard to say either way

2: Not very moisturized

1: Not moisturized

-Percent Occurrence of Split Ends and Broken Hair

Using as a standard tress a flattened hair tress styled with the hair foam of Comparative Example 2, ranking was performed in a similar manner as in Examples 1-3 and Comparative Example 1.

TABLE 2

| | | | | Comp. |
|---|---|---|---|---|
| (Wt. %) | Ex. 4 | Ex. 5 | Ex. 6 | Ex. 2 |
| Vinylpyrrolidone/N,N- | 2.5 | 2.5 | 2.5 | 2.5 |
| dimethylaminoethyl methacrylate | | | | |
| copolymer diethyl sulfate¹⁾ | | | | |
| Diamide compound (F) | 2.0 | 4.0 | 2.0 | — |
| Collagen hydrolysate²⁾ | — | — | 1.0 | — |
| Polyoxyethylene (9) | 1.0 | 1.0 | 1.0 | 1.0 |
| tridecyl ether | | | | |
| Ethanol | 10.0 | 10.0 | 10.0 | 10.0 |
| Purified water | Balance | Balance | Balance | Balance |
| Silkiness | 4.0 | 4.8 | 4.6 | 1.8 |
| Moisturized feel | 4.0 | 4.6 | 4.2 | 1.6 |
| Percent occurrence of split ends | 62 | 53 | 67 | 100 |
| and broken hairs | | | | |

³⁾“Gafquat 755N”, trade name; product of Kao Corporation
⁴⁾“PROMOIS E-118D”, trade name; product of Seiwa Chemical Industry Co., Ltd.

### EXAMPLE 7 & COMPARATIVE EXAMPLE 3

The “pump spray” liquids shown in Table 3 were formulated, and after applying to the hair for styling, evaluation was performed with regard to hair neatness.

<Evaluation Method>

With hair of Japanese subjects, flattened hair tresses of 26 cm in length, 0.5 cm in thickness and 15 g in weight were prepared. Each hair tress was evenly sprayed with the corresponding “pump spray” liquid at top, middle and bottom parts of both the front and rear sides of the tress, that is, at 6 locations in total, by pressing the pump down once at every location (approximately 0.4 g in total). The tress was combed, dried for 10 minutes in air, and then photographed from the side.

The thickness of the lower end of each tress was measured both before the treatment (dₒ) and after the treatment (d), and the voluminosity (d/dₒ×100) was calculated.

TABLE 3

| | | Comp. |
|---|---|---|
| (Wt. %) | Ex. 7 | Ex. 3 |
| Propylene glycol | 3.0 | 3.0 |
| Stearyltrimethylammonium chloride (28 wt. %) | 1.0 | 1.0 |
| Polyoxypropylene sorbitol | 1.0 | 1.0 |
| Polyoxyethylene hydrogenated castor oil | 0.5 | 0.5 |
| Polyoxyethylene tridecyl ether | 1.0 | 1.0 |
| Ethanol | 10.0 | 10.0 |
| Diamide compound (F) | 2.0 | — |
| Acrylamide/dimethyldiallylammonium | 1.5 | 1.5 |
| chloride copolymer¹⁾ | | |
| Deionized water | Balance | Balance |
| Voluminosity (%) | 46.3 | 61.0 |

¹⁾“MERQUAT 550”, trade name; product of Calgon Corp. (8.5 wt. % aqueous solution)

### EXAMPLE 8

### Aerosol Hair Spray

The materials shown below were mixed to provide a concentrate for an aerosol hair spray. After the concentrate was filled in a suitable pressure container, the pressure container was filled with a propellant (LPG, 2.5 kg/cm²) at a concentrate:propellant ratio (by weight) of 40:60 to obtain a hair spray.

| | (wt. %) |
|---|---|
| N-Methacryloyloxyethyl-N,N- | 12.0 |
| dimethylammonium-α-N-methylcarboxybetaine/ | |
| alkyl methacrylate copolymer¹⁾ | |
| N-propionyl polyethylenimine/ | 1.5 |
| methylpolysiloxane²⁾ | |
| Diamide compound (F) | 1.0 |
| Ceramide 3³⁾ | 0.5 |
| Methylphenylpolysiloxane | 0.5 |
| Perfume | q.s. |
| Ethanol | Balance |
| Total | 100.0 |

¹⁾“Yukaformer M-75”, trade name, product of Mitsubishi Chemical Corporation
²⁾“OS-96E”, trade name; product of Kao Corporation
³⁾“Ceramide III”, trade name; product of Gist-brocades/Cosmoferm BV

### EXAMPLE 9

### Hair Mist

The materials shown below were mixed to provide a concentrate for a hair mist, and then, the concentrate was filled in a suitable container.

| | (wt. %) |
|---|---|
| Vinyl methyl ether/ethyl maleate | 9.0 |
| copolymer¹⁾ | |
| Diamide compound (D) | 0.5 |
| Polyoxyethylene/methylpolysiloxane | 0.2 |
| copolymer²⁾ | |
| 2-Amino-2-ethyl-1-propanol | 0.4 |
| Perfume | q.s. |
| Purified water | 1.5 |
| Ethanol | Balance |
| Total | 100.0 |

¹⁾“Gantrez ES-225”, trade name; product of International Specialty Products Corporation
²⁾“Silicone L-7001”, trade name; product of Nippon Unicar Co., Ltd.

### EXAMPLE 10

### Brushing Aid

The materials shown below were mixed to provide a concentrate for a brushing aid, and then, the concentrate was filled in a suitable container.

| | (wt. %) |
|---|---|
| Hydroxypropyl chitosan liquid¹⁾ | 0.5 |
| Polyethylene glycol 20000 | 0.3 |
| Diamide compound (F) | 0.5 |
| Aminoethylaminopropylsiloxane/ | 1.0 |
| dimethylsiloxane copolymer emulsion²⁾ | |
| High-polymerization-degree | 1.0 |
| polymethylsiloxane emulsion³⁾ | |
| Stearyltrimethylammonium chloride | 0.2 |
| Ethanol | 25.0 |
| Purified water | Balance |
| Total | 100.0 |

¹⁾“Chitofilmer HV-10”, trade name, product of ICHIMARU PHARCOS Co., Ltd.
²⁾“SM8704C”, trade name; product of Dow Corning Toray Silicone Co., Ltd.
³⁾“Silicone KM-9716”, trade name; product of Shin-Etsu Chemical Co., Ltd.

### EXAMPLE 11

### Aerosol Foam (Soft Type) (pH. 7.0)

The materials shown below were mixed to provide a concentrate for a hair foam. After the concentrate was filled in a suitable pressure container, the container was filled with a propellant (LPG, 4.5 kg/cm²) at a concentrate:propellant ratio (by weight) of 90:10 to obtain an aerosol foam.

| | (wt. %) |
|---|---|
| Lauryl acrylate/vinyl acetate | 4.5 |
| copolymer (20 wt. %)¹⁾ | |
| Polyquartanium-11²⁾ | 3.0 |
| Polyquartanium-24³⁾ | 0.25 |
| Diamide compound (A) | 2.5 |
| Diamide compound (D) | 0.5 |
| Hydrolyzed silk liquid⁴⁾ | 0.1 |
| Aminoethylaminopropylsiloxane/ | 1.0 |
| dimethylsiloxane copolymer emulsion⁵⁾ | |
| Stearoxymethylpolysiloxane⁶⁾ | 0.5 |
| Sorbitol | 1.5 |
| Aluminum di-dl-pyrrolidonecarboxylate | 0.2 |
| (40 wt. %)⁷⁾ | |
| Polyoxyethylene tridecyl ether | 1.4 |
| Octyldodecanol | 0.8 |
| Methylparabene | 0.2 |
| Perfume | q.s. |
| Ethanol | 6.0 |
| Purified water | Balance |
| Total | 100.0 |

¹⁾“Polymer ND”, trade name; product of Kao Corporation
²⁾“Gafquat 755N-P”, trade name; product of ISP Japan Ltd.
³⁾“Quartery Soft Polymer LM-200”, trade name; product of Union Carbide Corporation
⁴⁾“Silkgen G Soluble KE”, trade name; product of ICHIMARU PHARCOS Co., Ltd.
⁵⁾“SM8704C”, trade name; product of Dow Corning Toray Silicone Co., Ltd.
⁶⁾“Silicone KF-7002”, trade name; product of Shin-Etsu Chemical Co., Ltd.
⁷⁾“PCA Alumi”, trade name; product of Kawaken Fine Chemicals Co., Ltd.

### EXAMPLE 12

### Aerosol Foam (Hard Type) (pH: 7.0)

The materials shown below were mixed to provide a concentrate for an aerosol foam, and the concentrate was filled in a suitable pressure container. Using as a propellant LPG (4.5 kg/cm²) and dimethyl ether at a weight ratio of 80:20, the container was filled with the propellant at a concentrate:propellant ratio (by weight) of 92:8 to obtain an aerosol foam.

| | (wt. %) |
|---|---|
| Carageenan¹⁾ | 1.5 |
| Alkyl acrylate/diacetone acrylamide | 0.5 |
| copolymer AMP²⁾ | |
| Diamide compound (A) | 2.0 |
| Methylphenylpolysiloxane³⁾ | 0.2 |
| Glycerin | 3.5 |
| Betaine | 0.5 |
| Sodium polyoxyethylene lauryl ether | 0.4 |
| phosphate⁴⁾ | |
| Polyoxyethylene tridecyl ether⁵⁾ | 0.4 |
| Diethylene glycol monoethyl ether | 1.0 |
| Methylparabene | 0.3 |
| Sodium monohydrogenphosphate | 0.25 |
| Citric acid | q.s. |
| Purified water | Balance |
| Total | 100.0 |

¹⁾“Soageena ML210”, trade name; product of MRC Polysaccharide Co., Ltd.
²⁾“PlassizeL-9540B”, tradename; product of GOO CHEMICAL CO., LTD.
³⁾“SILICONE SH-556”, trade name; product of Dow Corning Toray Silicone Co., Ltd.
⁴⁾“SPE-104NB”, trade name; product of Kao Corporation
⁵⁾“SOFTANOL 90”, trade name; product of Nippon Shokubai Co., Ltd.

### EXAMPLE 13

### Hair Blowing Aid

The materials shown below were combined into a uniform mixture to obtain a concentrate for a blowing aid, and the concentrate was filled in a suitable container.

| | (wt. %) |
|---|---|
| N-Methacryloyloxyethyl-N,N- | 0.5 |
| dimethylammonium-α-N-methylcarboxybetaine/ | |
| alkyl methacrylate copolymer¹⁾ | |
| Polyethylene glycol 20000 | 0.05 |
| Diamide compound (N) | 0.5 |
| Poloxyethylene/methylpolysiloxane | 0.3 |
| copolymer²⁾ | |
| Stearyltrimethylammonium | 1.0 |
| chloride (28 wt. %) | |
| Polyoxypropylene (9) sorbitol | 1.0 |
| Polyoxyethylene (9) tridecyl ether | 0.5 |
| Aluminum di-dl-pyrrolidonecarboxylate | 0.2 |
| solution (40 wt. %)³⁾ | |
| Birch extract⁴⁾ | 0.05 |
| Perfume | q.s. |
| Ethanol | 25.0 |
| Purified water | Balance |
| Total | 100.0 |

¹⁾“Yukaformer M-75”, trade name; product of Mitsubishi Chemical Corporation
²⁾“Silicone KF351A”, trade name; product of Dow Corning Toray Silicone Co., Ltd.
³⁾“PCA Alumi”, trade name; product of Kawaken Fine Chemicals Co., Ltd.
⁴⁾“Birch Extract”, trade name; product of ICHIMARU PHARCOS Co., Ltd.

### EXAMPLE 14

### Styling Gel (pH 8.0)

The materials shown below were uniformly mixed to obtain a thick concentrate for a styling gel, and the concentrate was filled in a suitable container.

| | (wt. %) |
|---|---|
| Alkyl acrylate/diacetone acrylamide | 4.0 |
| copolymer AMP¹⁾ | |
| Carageenan²⁾ | 1.2 |
| Polyvinylpyrrolidone³⁾ | 0.5 |
| Carboxyvinyl polymer⁴⁾ | 0.3 |
| Diamide compound (F) | 0.2 |
| Polyoxyethylene hydrogenated castor | 0.5 |
| oil⁵⁾ | |
| Polyoxyethylene lauryl ether⁶⁾ | 0.5 |
| Glycerin | 2.5 |
| Methylparabene | 0.2 |
| Perfume | q.s. |
| Propylene glycol | 2.5 |
| 48 wt. % sodium hydroxide | q.s. |
| Disodium edetate | 0.1 |
| Ethanol | 10.0 |
| Purified water | Balance |
| Total | 100.0 |

¹⁾“PlassizeL-9540B”, tradename; product of GOO CHEMICAL CO., LTD.
²⁾“Soageena LX-22”, trade name; product of MRC Polysaccharide Co., Ltd.
³⁾“Luviskol K-30”, trade name; product of BASF AG
⁴⁾“Carbopol”, trade name; product of BF Goodrich Co.
⁵⁾“Emanon CH-25”, trade name; product of Kao Corporation
⁶⁾“Emulgen 123P”, trade name; product of Kao Corporation

### EXAMPLE 15

### Styling Milk (pH Approx. 5.5 to 6.5)

The materials shown below were uniformly mixed to obtain a thick concentrate for a styling milk, and the concentrate was filled in a suitable container.

| | (wt. %) |
|---|---|
| Carageenan¹⁾ | 0.25 |
| Polyacrylamide-isoparaffin mixture²⁾ | 1.2 |
| Diamide compound (F) | 1.0 |
| Light liquid isoparaffin | 5.0 |
| Liquid paraffin | 6.0 |
| Cholesteryl isostearate | 0.25 |
| Trimethylglycerin | 0.5 |
| Methylparabene | 0.25 |
| Perfume | q.s. |
| 1,3-Butylene glycol | 5.0 |
| Ethanol | 7.5 |
| Purified water | Balance |
| Total | 100.0 |

¹⁾“Soageena LX-22”, trade name; product of MRC Polysaccharide Co., Ltd.
²⁾“Sepigel 305”, trade name; product of Seiwa Supply Co., Ltd.

1. A hair cosmetic composition comprising the following ingredients (A) and (B):
(A) a film-forming polymer; and
(B) a diamide compound represented by the following formula (1): 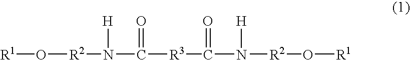
wherein R¹ represents a linear or branched C₁₋₁₂ hydrocarbon group which may be substituted by one or more hydroxyl groups and/or alkoxy groups, R²represents a linear or branched, divalent C₁₋₅ hydrocarbon group, and R³ represents a linear or branched, divalent C₁₋₂₂ hydrocarbon group.

2. A hair cosmetic composition according to claim 1, comprising as ingredient (B) a diamide compound of formula (1) in which R³ is a linear or branched C₁₁₋₂₂ alkylene group or a linear or branched C₁₁₋₂₂ alkenylene group having 1 to 4 double bonds.
